(19) United States
(12) Patent Application Publication
(10) Pub. No.: US 2026/0350793 A1
(43) Pub. Date: Nov. 5, 2026

(54) METHOD, DEVICE, AND COMPUTER PROGRAM FOR INTEGRATED PREDICTION OF DISEASE PROGNOSIS USING IMAGE AND NON-IMAGE DATA

(71) Applicant: CRESCOM CO., LTD., Seongnam-si, Gyeonggi-do (KR)

(72) Inventor: Jae Joon LEE, Seongnam-si (KR)

(73) Assignee: CRESCOM CO., LTD., Seongnam-si, Gyeonggi-do (KR)

(21) Appl. No.: 19/487,278

(22) PCT Filed: Jul. 19, 2024
(86) PCT No.: PCT/KR2024/010406

§ 371 Date: Nov. 24, 2025

(30) Foreign Application Priority Data
Jul. 20, 2023 (KR) 10-2023-0094629

(51) Int. Cl. G06T 7/00; G06T 7/11; G06V 10/25; G06V 10/82

(52) U.S. Cl. CPC G06T 7/0012; G06T 7/11; G06V 10/25; G06V 10/82; G06T 2207/20081

(57) ABSTRACT

A method for predicting the prognosis of a target disease on the basis of a medical image and non-image data according to an embodiment of the present invention may include: a step for acquiring a medical image of a patient being examined for an abnormal site; an image data analysis step for classifying the target disease in the medical image into a preset group by using a first artificial intelligence model; a first non-image data generation step for acquiring medical data on the patient and digitizing the same; a second non-image data generation step for digitizing target disease-related features in the medical image; and an integrated analysis step for predicting the prognosis of the target disease by applying a value obtained as a result of the image data analysis step and the first and second non-image data to a second artificial intelligence.

### TECHNICAL FIELD

The present invention relates to a method, device, and computer program for predicting a prognosis of a disease on the basis of medical image and non-image data, and more specifically, to a method, device, and computer program for analyzing and classifying an abnormal area included in a medical image, analyzing medical data of a subject, and integrating image and non-image data to predict a prognosis of a target disease.

### BACKGROUND ART

Currently, the problem of a shortage of doctors in Korea is becoming increasingly serious, and in particular, there is a problem of a lack of specialists in emergency medical sites, making it difficult to respond quickly.

By having doctors read medical images acquired by various medical diagnostic devices, such as radiography (or X-ray), ultrasound, computed tomography (CT), magnetic resonance imaging (MRI), and positron emission tomography (PET) images, earlier diagnosis and treatment of diseases are possible than in the past, and as a result, human lifetime expectancy is being realized. However, compared to the number of patients who use various diagnostic devices increases, the possibility of misdiagnosis caused by difficulties in timely diagnosis by specialists, inaccurate human interpretation, and interpretation differences between doctors or within the same doctor is also emerging as a problem. Further, the complex and time-consuming work processes in the medical sites place an excessive burden on medical staff. Moreover, while determining the degree of severity of a patient's condition and classifying treatment priorities are very important for timely treatment and saving lives, comprehensively analysis of complex data is necessary, and it is realistically very difficult to make immediate determination on complex data.

Therefore, various attempts are being made to overcome these problems by introducing artificial intelligence algorithm-based diagnostic systems in medical imaging. That is, research is actively being conducted to achieve more accurate diagnosis by supplementing the diagnostic findings using artificial intelligence (using second-opinion or double-reading) in addition to the interpretation of medical images by doctors. Further, artificial intelligence may aid to classify treatment priorities by predicting a patient's condition, severity, and prognosis.

However, in the same field of technology, attempts are being made to automatically interpret radiographs using machine learning models, but a method using machine learning models still has a limitation in accuracy, and due to the nature of machine learning, which is called a black box, there is a lack of explanation for results of automatic analysis, and thus it is rarely used in the sites.

### DESCRIPTION OF INVENTION

### Technical Problem

The present invention is intended to solve the above-described problems and aims to enable rapid and accurate prediction of a prognosis of a target disease on the basis of medical image and non-image data.

Further, the present invention also aims to improve improving the accuracy of predicting a prognosis of a target disease by comprehensively using image data and non-image data in medical image analysis using deep neural network artificial intelligence techniques.

### Technical Solution

The present invention is to achieve the above-described object, and a method of predicting a prognosis of a target disease on the basis of medical image and non-image data according to an embodiment of the present invention includes an operation of acquiring a medical image of a patient whose abnormal area is to be detected, an image data analysis operation of classifying the target disease within the medical image into a preset group using a first artificial intelligence model, an operation of generating first non-image data by acquiring medical data of the patient and quantifying the acquired medical data, an operation of generating second non-image data by quantifying features related to the target disease within the medical image, and an integrated analysis operation of predicting the prognosis of the target disease by applying a result value of the image data analysis operation and the first and second non-image data to a second artificial intelligence model.

### Advantageous Effects

According to the present invention, as described above, it is possible to rapidly and accurately predict a prognosis of a target disease using medical image and non-image data.

Further, according to the present invention, in medical image analysis using deep neural network artificial intelligence techniques, it is possible to improve the accuracy of classification of medical images.

### DESCRIPTION OF DRAWINGS

FIG. 1 is a schematic diagram of an electronic device according to an embodiment of the present invention.

FIGS. 2 to 4 are diagrams illustrating operations of an electronic device according to an embodiment of the present invention.

FIGS. 5 and 6 are diagrams illustrating artificial intelligence models of an electronic device according to an embodiment of the present invention.

FIGS. 7 and 8 are flowcharts illustrating a method of predicting a prognosis of a target disease according to an embodiment of the present invention.

### MODES OF THE INVENTION

The above-described objects, features, and advantages will be described in detail below with reference to the accompanying drawings, and accordingly, those skilled in the art to which the present invention pertains can easily implement the technical idea of the present invention. In describing the present invention, when detailed descriptions of well-known technologies related to the present invention are deemed to unnecessarily obscure the gist of the present invention, the detailed descriptions will be omitted. Hereinafter, exemplary embodiments of the present invention will be described in detail with reference to the accompanying drawings. In the drawings, the same reference numerals are used to indicate the same or similar components, and all combinations described in the specification and claims may be combined in any manner. In addition, unless otherwise described, a reference to a singular may include one or more, and it should be understood that a reference to a singular expression may also include a plural expression.

The present invention may be applied to various fields. In this specification, the content of the technology will be described with a focus on a method of predicting a prognosis of cerebral hemorrhage, degenerative arthritis, and/or growth and development abnormalities, but the present invention is not limited to the type of abnormal disease and may be utilized in various medical image analyses to determine abnormal conditions such as cardiovascular disease, cerebrovascular disease, etc.

Artificial intelligence models or integrated analysis models according to the present invention may be machine learning-based deep learning models. Further, the corresponding models may be trained using acquired data or statistical data.

Meanwhile, the term “abnormal area” used in this specification may be understood as a concept that includes not only an area determined to be abnormal in the binary classification of normal and abnormal, but also an area determined to be in various abnormal conditions (suspicion, cyst, tumor, fracture, fracture nonunion, acute fracture, bleeding, suspected bleeding, etc.) that are determined to be not normal.

Further, the term “abnormal condition” used in this specification may be understood as a term meaning the condition of an abnormal region present in a medical image or region of interest. The “abnormal condition” may include normal and abnormal, and more specifically, may include specific conditions of the abnormal area such as normal, suspicious, cystic, benign tumor, negative tumor, malignant tumor. Alternatively, when a medical image being analyzed is an image suspected of cerebral hemorrhage, the “abnormal condition” may include conditions such as normal, suspected cerebral hemorrhage, microbleed, hematoma enlargement, signs of increased intracranial pressure, and whether it is a supratentorial hemorrhage or a supratentorial hemorrhage.

Therefore, classification values that classify the target disease into preset groups may also be defined differently according to a classification method. For example, when an electronic device 1000 binary-classifies image data or non-image data (hereinafter referred to as data) into a preset group, i.e., normal or abnormal, data classified as normal according to the binary classification may have a classification value corresponding to something that is not an “abnormal condition,” and data classified as abnormal may have a classification value corresponding to an “abnormal condition.” When the electronic device 1000 multi-classifies image data or non-image data (hereinafter referred to as data), data classified as normal may have a classification value corresponding to something that is not an “abnormal condition,” data classified as suspicious may have a classification value where the type of “abnormal condition” corresponds to a “suspicious” category, data classified as having a water enchantment a classification value where the type of “abnormal condition” corresponds to a “water enchantment” category, data classified as having a water enchantment a classification value where the type of “abnormal condition” corresponds to a “water enchantment” category, and data classified as having a tumor may have a classification value where the type of “abnormal condition” corresponds to a “tumor” category. That is, the concept of the abnormal condition includes a plurality of condition types and may be understood as representing various conditions of an abnormal area, regions of interest (ROIs), and medical images, rather than simply a concept opposite to the normal condition.

Further, predicting a prognosis of cerebral hemorrhage according to the embodiment of the corresponding invention is a very important technology for providing appropriate treatment by predicting the prognosis of whether it is highly likely that this cerebral hemorrhage will expand and the hematoma volume will increase beyond a certain level when the cerebral hemorrhage occurs. Hematoma growth, which occurs in approximately 30% of spontaneous intracerebral hemorrhages, is closely related to mortality, and thus it is important to predict and provide appropriate treatment as a priority.

Further, predicting a prognosis of degenerative arthritis according to an embodiment of the corresponding invention is very important in order to provide a right treatment method depending on the area where degenerative arthritis occurs or the severity of the prognosis.

Further, the division of first to third artificial intelligence models in the present invention is merely for the purpose of enhancing understanding of the present invention, and in reality, there may be one model or a plurality of models.

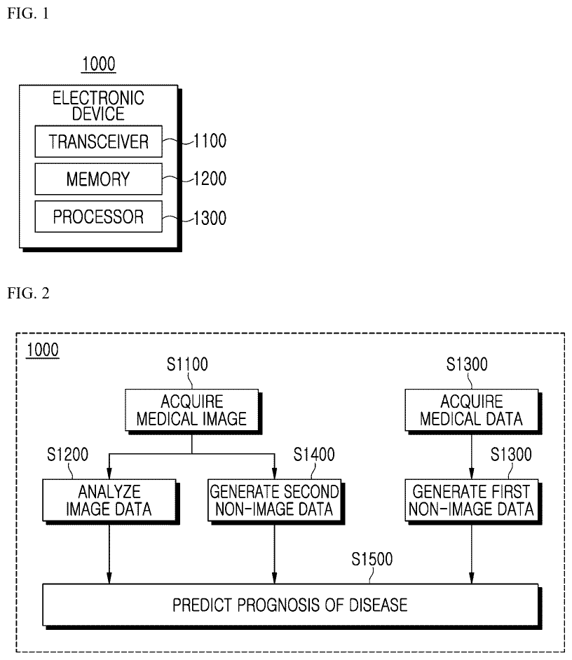

FIG. 1 is a schematic diagram of an electronic device 1000 according to an embodiment of the present invention.

The electronic device 1000 according to the embodiment of the present invention may include a transceiver 1100, a memory 1200, and a processor 1300.

The transceiver 1100 of the electronic device 1000 may communicate with a user terminal and/or any external device (or external server) including a database. For example, the electronic device 1000 may acquire an input requesting prediction of a prognosis of a disease from the user terminal or the external device through the transceiver 1100. For example, the electronic device 1000 may acquire medical data including a patient's sex, age, family medical history, genes, existing diseases, bone age information at the time of analysis, medication information, medical history information, height, weight, body temperature, lifestyle habits, and/or symptom information, which are necessary to predict the prognosis of the disease, from the user terminal through the transceiver 1100. For example, the electronic device 1000 may acquire disease prognosis statistical data classified by age, sex, country, region, and/or race from a database through the transceiver 1100. For example, the electronic device 1000 may transmit information on the predicted prognosis of the disease to any external device including a user terminal through the transceiver 1100.

As necessary, the electronic device 1000 may acquire medical images of the patient through the transceiver 1100. The medical images of the patient may be images of various body parts or images captured by various medical devices, according to a disease for which prognosis is to be predicted and may be acquired without restrictions on the body part or medical device.

The electronic device 1000 may access a network through the transceiver 1100 and transmit or receive various types of data. The transceiver 1100 may broadly include a wired type and a wireless type. Since the wired and wireless types each have their own advantages and disadvantages, the electronic device 1000 may be equipped with both a wired type and a wireless type in some cases. Here, in the case of a wireless type, communication methods based on wireless local area network (WLAN) family, such as Wi-Fi, may be used. Alternatively, in the case of a wireless type, cellular communication, such as Long-Term Evolution (LTE) or fifth generation (5G) series communication methods, may be used. However, the wireless communication protocols are not limited to the examples described above, and any appropriate wireless communication method may be used. In the case of a wired type, local area network (LAN) or Universal Serial Bus (USB) communication is representatively used, but other methods may also be used.

Various types of information may be stored in the memory 1200 of the electronic device 1000. Various types of data may be temporarily or semi-permanently stored in the memory 1200. Examples of the memory 1200 may include a hard disk drive (HDD), a solid state drive (SSD), a flash memory, a read-only memory (ROM), a random access memory (RAM), etc. The memory 1200 may be provided in the form of being embedded into or being detachably attached to the electronic device 1000. Various types of data required for the operation of the electronic device 1000, which include an operating program (e.g., operating system (OS)) for driving the electronic device 1000 and a program for operating each component of the electronic device 1000, may be stored in the memory 1200.

The processor 1300 may control the overall operation of the electronic device 1000. For example, the processor 1300 may control the overall operation of the electronic device 1000, which includes an operation of analyzing image data, an operation of generating and analyzing non-image data, and/or an operation of predicting a prognosis of a disease based on input data, which will be described below. Specifically, the processor 1300 may load and execute a program for the overall operation of the electronic device 1000 from the memory 1200. The processor 1300 may be implemented as an application processor (AP), a central processing unit (CPU), a microcontroller unit (MCU), or a device similar thereto, according to hardware, software, or a combination thereof. In this case, the processor 1300 may be provided in the form of an electronic circuit that processes electrical signals and performs a control function in terms of hardware and may be provided in the form of a program or code that drives a hardware circuit in terms of software.

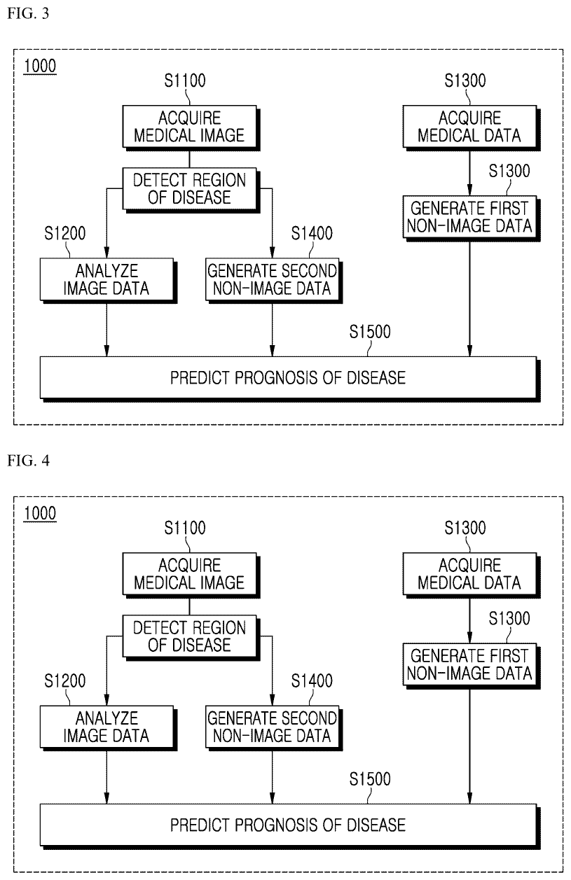

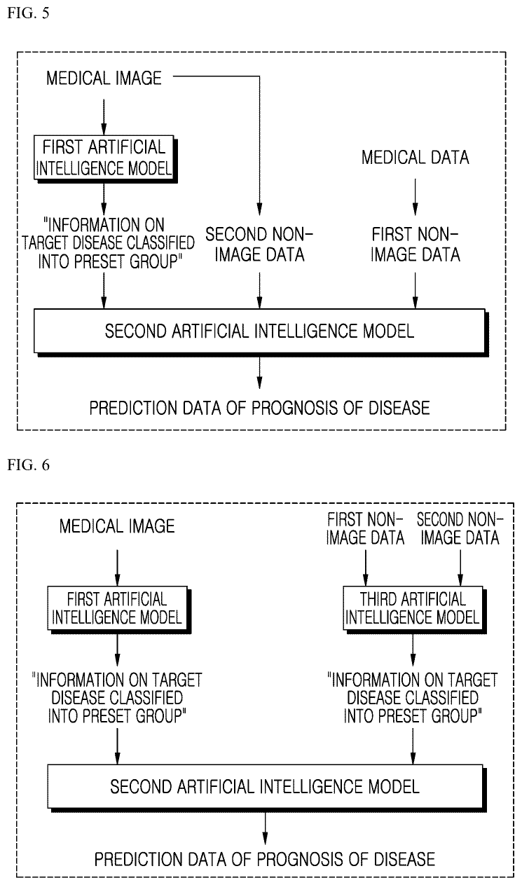

Hereinafter, the operation of an electronic device 1000 according to an embodiment of the present invention and a method of predicting a prognosis of a disease performed by the electronic device 1000 will be described in more detail with reference to FIGS. 2 to 8.

FIGS. 2 to 4 are diagrams illustrating operations of a processor 1300 according to the embodiment of the present invention.

Referring to FIGS. 2 to 4, the processor 1300 of the electronic device 1000 may be configured to acquire medical images and medical data of a patient whose abnormal area is to be detected, and to predict a prognosis of a disease using a generated integrated analysis model.

More specifically, according to an embodiment of the present invention, the processor 1300 of the electronic device 1000 may acquire input data through a transceiver 1100. Here, the input data may include medical images and medical data of the patient. More specifically, the medical images of the patient may be acquired through an imaging device such as a camera, an X-ray device, a computed tomography (CT) scan device, etc., or acquired through a capture function of the electronic device, and may include CT, X-ray, or magnetic resonance imaging (MRI) data of a specific region.

As necessary, the processor 1300 may preprocess the acquired image.

As necessary, the processor 1300 of the electronic device 1000 may extract one or more ROIs from a target image. Here, an ROI refers to a specific ROI that is important for analyzing the target image. In machine learning analysis, the importance of a specific region is often based on domain knowledge of the corresponding image. That is, analyzing centered on an ROI is to increase accuracy by removing noise in regions other than a main ROI that may potentially cause errors in the analysis.

As necessary, the processor 1300 of the electronic device 1000 may manually or automatically detect an ROI, for example, extract a region corresponding to location information set by a user, as the ROI.

As necessary, a first artificial intelligence model may automatically extract one or more ROIs from the target image. In machine learning analysis, the importance of a specific region is often based on domain knowledge of the corresponding image. That is, analyzing centered on the ROI is to increase accuracy by removing noise in regions other than a main ROI that may potentially cause errors in the analysis.

The first artificial intelligence model may include an automatic detection model, which is a deep neural network learned (trained and evaluated) to extract a plurality of ROIs from a target image. As the automatic detection model, detection techniques, such as a deep neural network-based convolutional neural network (CNN), a Faster-region-based CNN (R-CNN), You Only Look Once (YOLO), a transformer model, etc., may be used, but the present invention is not limited thereto.

As necessary, the processor 1300 of the electronic device 1000 may manually or automatically segment an image of the ROIs and extract regions related to a target disease from the segmented image. The purpose of extracting the ROIs is to improve accuracy, and the extraction of the ROIs is not done by analyzing the image using the entire image as input data, which is the case with conventional general image analysis methods, but rather enables for precise analysis of each major sub-region by performing machine learning modeling and application for each major sub-region.

As necessary, the processor 1300 of the electronic device 1000 may train an automatic detection model using a plurality of cerebral hemorrhage CT images and apply an arbitrary target image (brain CT) to the trained automatic detection model to extract one region corresponding to cerebral hemorrhage as an ROI. The automatic detection model may use a machine learning model trained with normal discrimination data and abnormal discrimination data that correspond to the ROI as learning data to detect an ROI according to whether an abnormal area is present within the ROI, segment an image of the ROI, and extract a region related to the target disease from the segmented image. The presence of an abnormal area within the ROI ultimately means the presence of an abnormal area within the target image, and thus it can be seen that the first artificial intelligence model determines and classifies the normal/abnormal condition of the target image. As an example, when the target image is a brain image and the first artificial intelligence model is a machine learning model trained with abnormal discrimination data indicating the presence of cerebral hemorrhage and normal discrimination data indicating the absence of cerebral hemorrhage, the electronic device may detect a region including a cerebral hemorrhage area within an input brain image as an ROI, segment an image of the ROI, and extract a region related to the target disease from the segmented image, or may determine whether the input image is positive for the target disease, and accordingly, classify an abnormal condition of the target image or an abnormal condition of the ROI.

As necessary, the processor 1300 of the electronic device 1000 may include a first artificial intelligence model that classifies a target disease within a medical image into a preset group. A machine learning model used in the first artificial intelligence model may be a deep neural network composed of one or more layers, and may be a convolution neural network (CNN) that includes a plurality of convolution layers that generate a feature map for features of an ROI and a pooling layer that performs subsampling between the plurality of convolution layers. The CNN may extract features from an input image by alternately performing convolution and subsampling on the input image.

The CNN may include multiple convolution layers and multiple subsampling layers (subsampling layers, max-pooling layers, and pooling layers) and include a global average pooling (GAP) layer, a fully-connected layer, a softmax layer, etc. The convolution layer is a layer that performs convolution on the input image, and the subsampling layer is a layer that extracts a maximum value or an average value locally from the input image and maps the extracted values to a two-dimensional (2D) image and may perform subsampling by making a local region larger. In the convolution layer, information, such as a size of a kernel, the number of kernels to be used (the number of maps to be generated), a weight table to be applied during the convolution operation, etc., is required. In the subsampling layer, information, such as a size of a kernel to be subsampled, whether a maximum value is selected or a minimum value is selected from among values within a kernel region, etc., is required. According to an embodiment, the CNN may include a pooling layer (Layer 2) in which subsampling is performed between convolution layers (Layer 1 and Layer 3) and may include a GAP layer, a fully-connected layer, and a softmax layer at the end. This is for convenience of description, the CNN may be formed by combining layers with different characteristics from those applied in this example in a more diverse manner, and the present invention is not limited by the configuration and structure of a deep neural network. The value output from the first artificial intelligence model is a value calculated by applying an ROI to a deep neural network machine learning model and means a classification value according to at least one of preset groups, and as described above, the classification value may be a value corresponding to each of normal/abnormal according to binary classification or a value corresponding to a class according to user settings, such as normal/suspicious/cyst/tumor (negative, benign, and malignant) or normal/suspicious fracture/fracture nonunion/acute fracture/fracture according to multi-classification.

As necessary, the processor 1300 of the electronic device 1000 may be a model trained to classify the target disease in the medical image into the preset group using the first artificial intelligence model. The preset group includes one or more groups including a disease type group, a disease severity grade group, a group indicating whether a disease is positive, a disease prognosis prediction group, and a cerebral hematoma enlargement prediction group. That is, in the case of a brain image, the preset group includes a group classified by a disease such as stroke or cerebral hemorrhage, a group classified by a plurality of disease severity grades, a group classified by binary classification of whether the target disease is positive or negative, and a group classified by a prognosis of a disease, and the electronic device may classify the medical image of the patient into one or more of these groups among the above groups using the first artificial intelligence model.

According to a specific embodiment of the present invention, the electronic device may apply a brain CT image to the first artificial intelligence model to quantify and output the possibility that a volume of cerebral hemorrhage will increase by a certain amount or more.

The electronic device may divide the image of the extracted region related to the target disease into normal discrimination data and abnormal discrimination data to train the first artificial intelligence model on the basis of the normal discrimination data and the abnormal discrimination data.

As necessary, the processor 1300 of the electronic device 1000 may quantify the medical data of the patient to generate first non-image data. The medical data may be one or more pieces of data selected from a group including the patient's age, sex, family medical history, genes, underlying diseases, existing diseases, medications, height, weight, body temperature, lifestyle habits, and symptoms, and may also include any information that may affect the prognosis of the disease.

The method of quantifying the medical data may be achieved through various methods, such as a method of quantifying the medical data by allowing a user to assign weights to factors affecting the prognosis of the disease, a method set by the user, a Glasgow Coma Scale (GCS) method, etc., and the present invention is not limited to those described.

As necessary, the processor 1300 of the electronic device 1000 may quantify features related to the target disease within the medical image to generate second non-image data. The features related to the target disease may include all features among patient information included in the image that may affect the prognosis of the target disease, and the second non-image data refers to data obtained by quantifying the locations, volumes, and shapes of the features related to the disease, etc. included in the image.

As necessary, the processor 1300 of the electronic device 1000 may quantify features related to the target disease using intermediate output values of the first artificial intelligence model to generate second non-image data. More specifically, the intermediate output values may be values obtained by extracting the image of the ROI detected through the first artificial intelligence model or the region related to the target disease after segmenting the image of the ROI. The processor may generate more accurate data by quantifying the region related to the disease using the intermediate output values of the first artificial intelligence model.

In an embodiment, when an image related to cerebral hemorrhage is acquired, the processor 1300 of the electronic device 1000 may quantify the thickness of blood vessels, the location coordinates of the cerebral hemorrhage area, three-dimensional (3D) coordinates of the center point of the cerebral hemorrhage, the relative location within the brain region, the volume and area of the cerebral hemorrhage area, the area and location of the largest cerebral hemorrhage, the number and volume of each type of cerebral hemorrhage, the image patterns and texture characteristics of the cerebral hemorrhage region, the shape and condition of the cerebral hemorrhage, etc. to generate second non-image data.

According to another embodiment, when acquiring an image related to degenerative arthritis, the processor 1300 of the electronic device 1000 may quantify a joint spacing, a hardening location, a hardening degree, an osteophyte location, an osteophyte degree, a bone number, a bone spacing, a bone thickness, etc. of a target region within the image to generate second non-image data.

As necessary, the processor 1300 of the electronic device 1000 according to the embodiment of the present invention may classify first and second non-image data into preset groups by applying the first and second non-image data to a third artificial intelligence model. The third artificial intelligence model may analyze the first and second non-image data using machine learning techniques such as support vector machine (SVM), regression, a fully connected neural network model, etc., and perform the desired double classification, multi-classification, etc. In this case, since the preset group is the same as the one described above, a detailed description thereof will be omitted.

The processor 1300 of the electronic device 1000 according to the embodiment of the present invention may generate a second artificial intelligence model for comprehensively predicting a prognosis of a disease on the basis of the medical image and non-image data.

The electronic device 1000 according to the embodiment of the present invention may acquire prognostic statistical data of a target disease through the transceiver 1100. The prognostic statistical data may be grouped by location, volume, age, sex, country, region, and/or race of the target disease. The electronic device 1000 according to the embodiment of the present invention may train the second artificial intelligence model capable of predicting the prognosis of the target disease on the basis of input data and the prognostic statistical data of the disease.

As necessary, the processor 1300 of the electronic device 1000 may predict the prognosis of the target disease from medical images and medical data of the patient using the second artificial intelligence model. More specifically, the electronic device 1000 may predict the prognosis of the target disease including weights (or parameters) derived through the artificial intelligence model, as described above.

According to an embodiment of the present invention, the second artificial intelligence model may output the predicted value of the prognosis of the target disease using the output value of the first artificial intelligence model and the first and second non-image data as the input data.

As necessary, the second artificial intelligence model may output a prediction value of the prognosis of the target disease using the output values of the first artificial intelligence model and the output values of the third artificial intelligence model as the input data.

As necessary, the artificial intelligence models according to the embodiment of the present invention may use the scored or normalized probability valued of each class for the final class classification of the deep neural network as classification values. For example, the class score values of each softmax layer or the normalized probability values resulting from the softmax layer may be calculated as classification values for the corresponding region of interest and input into the artificial intelligence models.

$${S}^{c} = {\sum }_{i}^{K} {w}_{i}^{c} {A}^{i} \qquad [ Equation 1 ]$$

Here, S^{C} denotes a score of class C, and w^{c}ᵢ denotes a weight of Aⁱ.

$${A}^{k} = \frac{1}{{\sum }_{i} {\sum }_{j} 1} {\sum }_{i} {\sum }_{j} {M}_{i , j}^{k} \qquad [ Equation 2 ]$$

A^{k} denotes an average value of feature values M^{k} in channel k, and in this case, a feature value of a region h of interest may be composed of Fh=[S1, S2, S3, . . . , and SM]. M denotes a total number of classes.

The processor 1300 of the electronic device 1000 according to the embodiment of the present invention may generate an integrated prediction model for predicting a prognosis of a target disease. Furthermore, the processor 1300 of the electronic device 1000 may be configured to perform an operation to predict the prognosis of the target disease from input data including medical images of the patient and/or medical data of the patient using the generated integrated prediction model. Further, the processor 1300 of the electronic device 1000 may be configured to perform an operation to predict the prognosis of the target disease by applying the medical images of the patient and medical image and non-image data obtained by processing the medical data to the generated integrated prediction model.

According to a specific embodiment of the present invention, the electronic device 1000 may acquire brain CT medical images and medical data of a patient suspected of having a cerebrovascular disease. The electronic device 1000 may apply the medical images of the patient to the first artificial intelligence model as input data, classify the type of cerebrovascular disease of the patient, and when it is determined that there is a high possibility of cerebral hemorrhage hematoma hypertrophy, classify the medical image of the patient into a group corresponding to the cerebral hemorrhage hematoma hypertrophy.

In addition, the electronic device 1000 may specify coordinates (x, y, z) of a center point of the cerebral hemorrhage through the medical image of the patient to quantify the center point and may quantify a volume (e.g., mL) of the cerebral hemorrhage region and store the quantified data as the second non-image data. Since the risk of cerebral hemorrhage may vary depending on the coordinates of the center point, the volume of the cerebral hemorrhage, etc., the prognosis of the target disease may be more accurately predicted by quantifying the corresponding data and using the quantified data for analysis. The corresponding data may be generated in the form of scores and stored in the form of a vector to be input into the second artificial intelligence model.

Further, the electronic device 1000 may quantify the medical data to generate first non-image data. In an embodiment, according to the Glasgow Coma Scale, the severity of the patient's condition may be determined by scoring the patient's eye response level as from 1 to 4, the verbal response level as from 1 to 5, and the motor response level as from 1 to 6.

According to another embodiment, the medical data of the patient may be scored according to the target disease by scoring the patient's age group, scoring the risk according to the patient's sex, and when the patient has an underlying disease, assigning the degree of risk according to the underlying disease. That is, in the case in which the patient has cerebral hemorrhage, the degree of risk may be scored as 5 when the patient is over 60 years old, as 4 when the patient is in his/her 50s, and as 3 when the patient is in his/her 40s, and the first non-image data may be generated by quantifying the medical data of the patient by adding the degree of risk of 5 when the patient has high blood pressure and the degree of risk of 20 when the patient already has a history of cerebral hemorrhage.

The electronic device may input the first and second non-image data into the third artificial intelligence model and classify the target disease into the preset group. The electronic device may determine that the patient is less likely to have a cerebral hemorrhage or hematoma enlargement through the patient's first and second non-image data.

The second artificial intelligence model of the electronic device may use the image data and the first and second non-image data as input data to predict the prognosis of the disease.

As necessary, the second artificial intelligence model may use output values of the first and third artificial intelligence model as input data to predict the prognosis of the disease. When the groups of the diseases classified through the first and third artificial intelligence model are different, the possibility of predicting the prognosis may be further improved by additionally determining the prognosis through the second artificial intelligence model.

Hereinafter, a method of predicting a prognosis of a target disease on the basis of medical image and non-image data corresponding to still another embodiment of the present invention will be described.

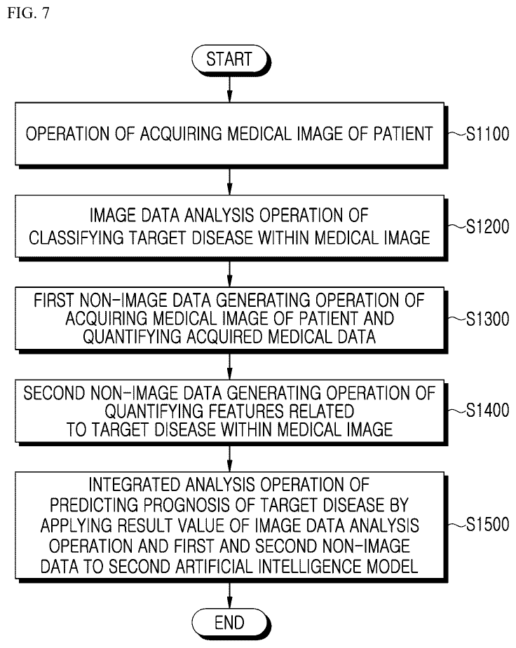

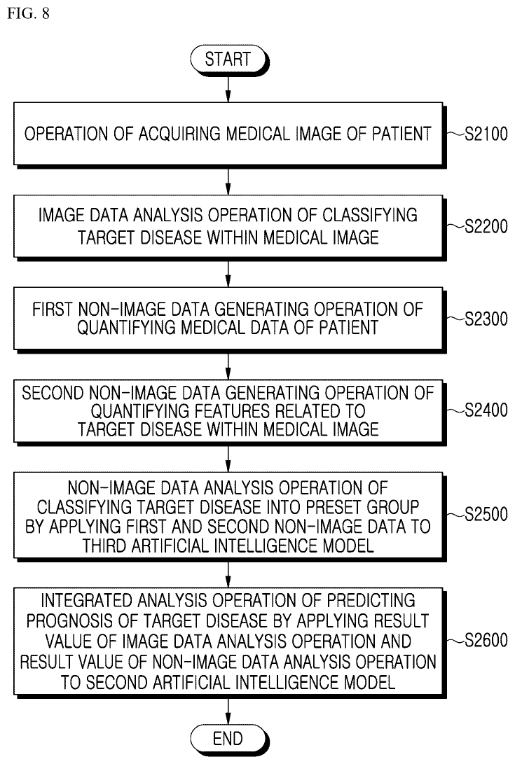

FIGS. 7 and 8 are flowcharts of methods of predicting a prognosis of a target disease according to embodiments of the present invention.

The method of predicting the prognosis of the target disease according to the embodiment of the present invention relates to a method of comprehensively predicting a prognosis of a disease by acquiring image and non-image data of a patient and using results of analyzing the acquired image and non-image data individually or integrated, and relates to a method of more accurately predicting a prognosis of a target disease.

A method of predicting a prognosis of a target disease according to a first embodiment of the present invention may include an operation S1100 of acquiring a medical image of a patient whose abnormal area is to be detected, an image data analysis operation S1200 of classifying the target disease within the medical image into a preset group using a first artificial intelligence model, an operation S1300 of generating first non-image data by acquiring medical data of the patient and quantifying the acquired medical data, an operation S1400 of generating second non-image data by quantifying features related to the target disease within the medical image, and an integrated analysis operation S1500 of predicting the prognosis of the target disease by applying a result value of the image data analysis operation and the first and second non-image data to a second artificial intelligence model.

A method of predicting a prognosis of a target disease according to a second embodiment of the present invention may include an operation S2100 of acquiring a medical image of a patient whose abnormal area is to be detected, an image data analysis operation S2200 of classifying the target disease within the medical image into a preset group using a first artificial intelligence model, an operation S2300 of generating first non-image data by acquiring medical data of the patient and quantifying the acquired medical data, an operation S2400 of generating second non-image data by quantifying features related to the target disease within the medical image, a non-image data analysis operation S2500 of classifying the target disease into the preset group by applying the first and second non-image data to a third artificial intelligence model, and an integrated analysis operation S2600 of comprehensively predicting the prognosis of the target disease by applying a result value of the image data analysis operation and a result value of the non-image data analysis operation to a second artificial intelligence model.

As necessary, in the methods of predicting the prognosis of the target disease according to the first and second embodiments of the present invention, the image data analysis operation may include an operation of detecting an ROI containing the abnormal area from the medical image acquired using the first artificial intelligence model, an operation of segmenting an image of the ROI, and an operation of extracting a region related to the target disease from the segmented image. As necessary, the first artificial intelligence model may extract the region related to the target disease and then classify the target disease in the extracted image into the preset group.

As necessary, the operation of generating the first non-image data may include an operation of scoring the medical data. The medical data may include one or more pieces of data from a group including the patient's age, sex, family medical history, genes, underlying diseases, pre-existing diseases, medications, body temperature, height, weight, body temperature, lifestyle habits, and symptoms.

As necessary, the operation of generating the second non-image data may include an operation of quantifying features of the region related to the target disease. As necessary, the electronic device may generate the second non-image data by quantifying the features of the region related to the target disease using the data extracted from the region related to the target disease within the segmented image.

As necessary, in the methods according to the first and second embodiments of the present invention, the preset group may include one or more groups including a disease type group, a disease severity grade group, a group indicating whether a disease is positive, a disease prognosis prediction group, and the features related to the target disease may include one or more features from a group including a location, a size, and a volume of the region related to the target disease, and physical features associated with the target disease.

As necessary, in the methods of predicting the prognosis of the target disease according to the first and second embodiments of the present invention, the integrated analysis operation may include an operation of predicting a prognosis of degenerative arthritis or growth and development of the body.

As necessary, in the methods of predicting the prognosis of the target disease according to the first and second embodiments of the present invention, the integrated analysis operation may include an operation of predicting a prognosis of cerebral hemorrhage hematoma hypertrophy.

In still another embodiment of the present invention, a computer-readable recording medium on which a program for executing the method of predicting the prognosis of the target disease of the present invention may be provided.

The features, structures, effects, etc., described in the embodiments above are included in at least one embodiment of the present invention and are not necessarily limited to only one embodiment. Furthermore, the features, structures, effects, etc. exemplified in each embodiment may be combined or modified and implemented in other embodiments by those skilled in the art to which the embodiments belong. Therefore, the content related to such combinations and modifications should be interpreted as being included in the scope of the present invention. Further, while the present invention has been particularly described with reference to embodiments, the embodiments are only exemplary embodiments of the present invention and the present invention is not intended to be limited thereto. It will be understood by those skilled in the art that modifications and applications in other forms may be made without departing from the spirit and scope of the present invention. That is, each component specifically shown in the embodiments may be modified and embodied. In addition, it should be understood that differences related to these modifications and applications are within the scope of the present invention as defined in the appended claims.

1. A method of predicting a prognosis of a target disease, which is a method of predicting a prognosis of a target disease on the basis of medical image and non-image data, the method comprising:
an operation of acquiring a medical image of a patient whose abnormal area is to be detected;
an image data analysis operation of classifying the target disease within the medical image into a preset group using a first artificial intelligence model;
an operation of generating first non-image data by acquiring medical data of the patient and quantifying the acquired medical data;
an operation of generating second non-image data by quantifying features related to the target disease within the medical image; and
an integrated analysis operation of predicting the prognosis of the target disease by applying a result value of the image data analysis operation and the first and second non-image data to a second artificial intelligence model.

2. A method of predicting a prognosis of a target disease, which is a method of predicting a prognosis of a target disease on the basis of medical image and non-image data, the method comprising:
an operation of acquiring a medical image of a patient whose abnormal area is to be detected;
an image data analysis operation of classifying the target disease within the medical image into a preset group using a first artificial intelligence model;
an operation of generating first non-image data by acquiring medical data of the patient and quantifying the acquired medical data;
an operation of generating second non-image data by quantifying features related to the target disease within the medical image;
a non-image data analysis operation of classifying the target disease into the preset group by applying the first and second non-image data to a third artificial intelligence model; and
an integrated analysis operation of comprehensively predicting the prognosis of the target disease by applying a result value of the image data analysis operation and a result value of the non-image data analysis operation to a second artificial intelligence.

3. The method of claim 1, wherein the image data analysis operation includes:
an operation of detecting a region of interest (ROI) containing the abnormal area from the medical image;
an operation of segmenting an image of the ROI; and
an operation of extracting a region related to the target disease from the segmented.

4. The method of claim 3, wherein the operation of generating the second non-image data includes an operation of quantifying features of the region related to the target disease.

5. The method of claim 1, wherein the preset group includes one or more groups including a disease type group, a disease severity grade group, a group indicating whether a disease is positive, and a disease prognosis prediction group, and
the features related to the target disease includes one or more features from a group including a location, a size, and a volume of the region related to the target disease, and physical features associated with the target.

6. The method of claim 1, wherein the medical data includes one or more pieces of data from a group including the patient's age, sex, family medical history, genes, underlying diseases, pre-existing diseases, medications, body temperature, height, weight, body temperature, lifestyle habits, and symptoms, and
the operation of generating the first non-image data includes an operation of scoring the medical.

7. The method of claim 1, wherein the integrated analysis operation includes an operation of predicting a prognosis of degenerative arthritis or growth and development of the body.

8. The method of claim 1, wherein the integrated analysis operation includes an operation of predicting a prognosis of cerebral hemorrhage hematoma hypertrophy.

9. A computer-readable recording medium on which a program for executing the method according to claim 1 on a computer is recorded.

10. An electronic device for predicting a prognosis of a target disease on the basis of medical image and non-image data, the electronic device including a processor configured to:
acquire a medical image of a patient whose abnormal area is to be detected;
classify the target disease within the medical image into a preset group using a first artificial intelligence model;
generate first non-image data by acquiring medical data of the patient and quantifying the acquired medical data of the patient;
generate second non-image data by quantifying features related to the target disease within the medical image; and
comprehensively predict the prognosis of the target disease by applying an output value of the first artificial intelligence model and the first and second non-image data to a second artificial intelligence.

11. An electronic device for predicting a prognosis of a target disease on the basis of medical image and non-image data, the electronic device including a processor configured to:
acquire a medical image of a patient whose abnormal area is to be detected;
classify the target disease within the medical image into a preset group using a first artificial intelligence model;
generate first non-image data by acquiring medical data of the patient and quantifying the acquired medical data of the patient;
generate second non-image data by quantifying features related to the target disease within the medical image;
classify the target disease into the preset group by applying the first and second non-image data to a third artificial intelligence model; and
comprehensively predict the prognosis of the target disease by applying output values of the first and third artificial intelligence models to a second artificial intelligence.
